(19) United States
(12) Patent Application Publication
(10) Pub. No.: US 2025/0132061 A1
(43) Pub. Date: Apr. 24, 2025

(54) ONLINE DISPENSING SYSTEM, INFORMATION LINKAGE SERVER, ONLINE DISPENSING METHOD, AND PROGRAM

(71) Applicant: NIPPON TELEGRAPH AND TELEPHONE CORPORATION, Tokyo (JP)

(72) Inventors: Takefumi MOGAKI, Musashino-shi (JP); Koji KOREKAWA, Musashino-shi (JP)

(73) Assignee: NIPPON TELEGRAPH AND TELEPHONE CORPORATION, Tokyo (JP)

(21) Appl. No.: 18/691,520

(22) PCT Filed: Sep. 30, 2021
(86) PCT No.: PCT/JP2021/036123

§ 371 Date: Mar. 13, 2024

(51) Int. Cl. G16H 70/40

(52) U.S. Cl. CPC G16H 70/40

(57) ABSTRACT

An online dispensing system includes a first information acquisition unit configured to acquire first information on side effects of a medicine to be delivered to a user on the basis of medicine information on the medicine written in a prescription of the user and genetic information of the user, a linkage processor configured to provide the first information to a pharmacist terminal device used by a pharmacist as side effect information on the medicine, and a pharmacist terminal device configured to support execution of administration instructions through communication with a user terminal device used by the user and to enable reference to the side effect information in the administration instructions, with respect to the administration instructions of the medicine executed for the user when the pharmacist delivers the medicine.

### TECHNICAL FIELD

The present invention relates to a technique for performing a dispensing service online.

### BACKGROUND ART

Various attempts have been made using ICT in order to reduce contact between people and people in the case of medicine delivery with the world epidemic of COVID 19 as a background. For example, a user who receives medicine delivery applies online administration instructions from an application of a smartphone, a pharmacist prepares medicine on the basis of data of a prescription issued by a doctor and delivers the medicine to the user in a dispensing pharmacy, and the pharmacist performs administration instructions for the user online (refer to Non Patent Literature 1, for example).

### CITATION LIST

### Non Patent Literature

[NPL 1] “What you can do with CLINICS application,” Medley, Internet <URL: https://clinics.medley.life/guide>.

### SUMMARY OF INVENTION

### Technical Problem

However, in the case of online administration instructions, communication with a user becomes complicated as compared to the case of performing administration instructions in a face-to-face manner, and thus the amount of information exchanged with the user is reduced and the quality of administration instructions may be deteriorated.

In view of the foregoing, an object of the present invention is to provide a technique capable of improving the quality of online administration instructions.

### Solution to Problem

One aspect of the present invention is an online dispensing system including: a first information acquisition unit configured to acquire first information on side effects of a medicine to be delivered to a user on the basis of medicine information on the medicine written in a prescription of the user and genetic information of the user; a linkage processor configured to provide the first information to a pharmacist terminal device used by a pharmacist as side effect information on the medicine; and a pharmacist terminal device configured to support execution of administration instructions through communication with a user terminal device used by the user and to enable reference to the side effect information in the administration instructions, with respect to the administration instructions of the medicine executed for the user when the pharmacist delivers the medicine.

One aspect of the present invention is an information linkage server including: a genetic information server configured to store genetic information of a user; a pharmacist terminal device used by a pharmacist who performs administration instructions with respect to a medicine to be delivered to the user; and a linkage processor configured to perform linkage of various types information with a user terminal device used by the user, in which the linkage processor acquires medicine information on a medicine written in a prescription of the user from the pharmacist terminal device, provides the medicine information to the genetic information server, and provides information on side effects of the medicine generated by the genetic information server on the basis of the medicine information to the pharmacist terminal device.

One aspect of the present invention is an online dispensing method including: a first information acquisition step of acquiring first information on side effects of a medicine to be delivered to a user on the basis of medicine information on the medicine written in a prescription of the user and genetic information of the user; and an information linkage step of providing the first information to a pharmacist terminal device used by a pharmacist as side effect information on the medicine, in which the pharmacist terminal device supports execution of administration instructions through communication with a user terminal device used by the user and enables reference to the side effect information in the administration instructions, with respect to the administration instructions of the medicine executed for the user when the pharmacist delivers the medicine.

One aspect of the present invention is a program causing a computer communicating with a genetic information server configured to store genetic information of a user, a pharmacist terminal device used by a pharmacist who performs administration instructions with respect to a medicine to be delivered to the user, and a user terminal device used by the user to: acquire medicine information on a medicine written in a prescription of the user from the pharmacist terminal device and provide the medicine information to the genetic information server; and provide information on side effects of the medicine generated by the genetic information server on the basis of the medicine information to the pharmacist terminal device.

### Advantageous Effects of Invention

According to the present invention, the quality of online administration instructions can be improved.

### BRIEF DESCRIPTION OF DRAWINGS

FIG. 1 is a diagram illustrating an example of a configuration of an online dispensing system according to a first embodiment.

FIG. 2 is a diagram illustrating an example of a functional configuration of an information linkage server according to the first embodiment.

FIG. 3 is a flowchart illustrating an example of a flow of preprocessing performed by a genetic information server.

FIG. 4 is a diagram illustrating a specific example of a medicine metabolism list generated by preprocessing.

FIG. 5 is a diagram illustrating an example of other information related to a metabolic rate.

FIG. 6 is a flowchart illustrating an example of a flow of main processing performed by the genetic information server.

FIG. 7 is a flowchart illustrating an example of a flow of processing in which a medical checkup information server generates medical checkup data.

FIG. 8 is a sequence diagram illustrating an example of a flow of processing executed by each server and each terminal device in the online dispensing system according to the first embodiment with respect to online administration instructions.

FIG. 9 is a diagram illustrating an example of a screen of a pharmacist application operating on a pharmacist terminal device.

FIG. 10 is a diagram illustrating an example of a screen of a user application operating on a user terminal device.

FIG. 11 is a diagram illustrating an example of a configuration of an online dispensing system according to a second embodiment.

FIG. 12 is a diagram illustrating an example of a functional configuration of an information linkage server according to the second embodiment.

FIG. 13 is a flowchart illustrating an example of a flow of processing performed by a medicine combination server.

FIG. 14 is a sequence diagram illustrating an example of a flow of processing executed by each server and each terminal device in the online dispensing system according to the second embodiment with respect to online administration instructions.

### DESCRIPTION OF EMBODIMENTS

Embodiments of the present invention will be described in detail with reference to the drawings.

### First Embodiment

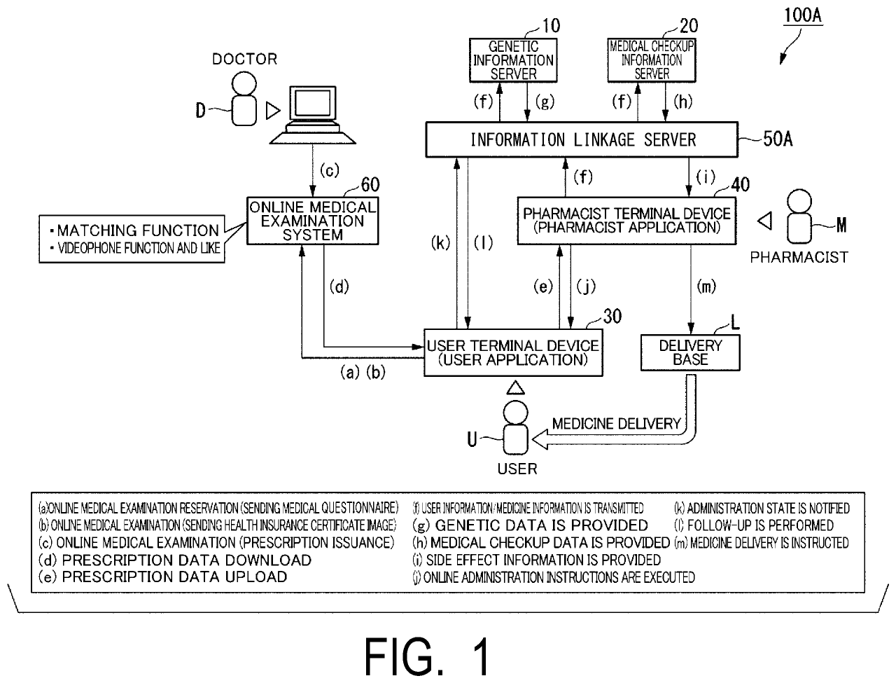

FIG. 1 is a diagram illustrating an example of a configuration of an online dispensing system 100A according to a first embodiment of an administration instructions system of the present invention. The online dispensing system 100A provides an online dispensing service to a patient (hereinafter referred to as a “user”) who has a prescription issued by a doctor. The online dispensing service provides an online a service in which a pharmacist compounds and delivers a medicine based on a prescription of a user. The online dispensing service includes administration instructions to be executed at the time of delivering a medicine compounded by a pharmacist to a user, and the online dispensing service also executes the administration instructions online. Hereinafter, administration instructions executed online is referred to as “online administration instructions.” Administration instructions executed by the pharmacist include instructions on basic items such as administration timing of a medicine, the number of times of administration, and a dosage, and instructions on storage methods, side effects that require cautions, medicine combination, and the like.

The online dispensing system 100A includes, for example, a genetic information server 10, a medical checkup information server 20, a user terminal device 30, a pharmacist terminal device 40, and an information linkage server 50A. The information linkage server 50A can communicate with the genetic information server 10, the medical checkup information server 20, and the pharmacist terminal device 40. The user terminal device 30 can communicate with the pharmacist terminal device 40 and an online medical examination system 60.

Here, the online medical examination system 60 is a system for supporting execution of an online medical examination. In the present embodiment, it is assumed that a doctor D examines a user U using the online medical examination system 60 and issues a prescription of a medicine according to diagnostic results as electronic data. Hereinafter, electronic data of a prescription is referred to as “prescription data.” Further, in the present embodiment, it is assumed that “prescribing” means that the doctor D indicates a medicine to be delivered by a prescription to the user U or that a pharmacist M is instructed to prepare the medicine to be delivered to the user U according to issuance of the prescription.

The genetic information server 10 has a function of generating information on side effects of the medicine prescribed for the user U by the doctor D (hereinafter referred to as “genetic data”) on the basis of genetic information of the user U and providing the generated genetic data to the information linkage server 50A. The genetic information is information indicating results of a genetic test and the constitution of the user U obtained on the basis of the result of the genetic test. For example, the genetic information is obtained by analyzing the blood of the user U collected in a hospital or the like by a genotyping agent. For example, a genotyping agent mainly detects differences in types in which DNA sequences differ by only one base, referred to as single nucleotide polymorphism (SNP), by a microarray or the like. Genotyping may be directed to the whole genome, or may be directed to a specific region in the whole genome. Genotyping may be selected according to required genetic information. In the present embodiment, it is assumed that the genetic information server 10 holds genetic information with respect to the user U of the online dispensing system 100A in advance. The genetic information server 10 is an example of a “first information acquisition unit.” Genetic data is an example of “first information.”

The medical checkup information server 20 has a function for providing medical checkup data of the user U to the information linkage server 50A. The medical checkup data is data indicating health problems, precautions, and the like that can be detected from the health condition of the user U and is generated on the basis of information indicating result of medical checkup of the user U (hereinafter referred to as “medical checkup information”), for example. In the present embodiment, it is assumed that the medical checkup information server 20 holds medical checkup information indicating result of medical checkup with respect to the user U of the online dispensing system 100A in advance. The medical checkup information server 20 is an example of a “second information acquisition unit.” Medical checkup data is an example of “second information.”

The user terminal device 30 is a terminal device used for the user U of the online dispensing system 100A to receive online administration instructions from the pharmacist M. The user terminal device 30 is, for example, a portable terminal device such as a smartphone or a tablet. Specifically, the user terminal device 30 has a function of receiving prescription data from the online medical examination system 60 as input, providing the input prescription data to the pharmacist terminal device 40, and allowing the user U to receive online administration instructions in cooperation with the pharmacist terminal device 40. Further, the user terminal device 30 has a function of notifying the information linkage server 50A of an administration state of the user U after delivery of a medicine and receiving follow-up based on the administration state of the user U from the information linkage server 50A. These functions of the user terminal device 30 are realized, for example, by the user terminal device 30 executing an application program (hereinafter referred to as “user application”). Follow-up is an example of “supplementary information.”

Note that the user application may have a function of supporting medical examination of the user U by the online medical examination system 60. For example, the user application may have a matching function of linking with the online medical examination system 60 and introducing the user U to the doctor D, a function of providing a means (for example, a voice call function, a videophone function, or the like) for communication with the doctor D, a function of linking various types of information (for example, a medical questionnaire, a health insurance card image, and the like) necessary for medical examinations, and the like. The user application may be installed in the user terminal device 30 as a dedicated application, or may be realized by a general-purpose application such as a browser.

The pharmacist terminal device 40 is a terminal device used for the pharmacist M to execute administration instructions and subsequent follow-up or the like online in delivering a medicine to the user U. The pharmacist terminal device 40 is, for example, a terminal device such as a PC, a smartphone, or a tablet. The pharmacist terminal device 40 is installed, for example, in a store of a dispensing pharmacy.

Specifically, the pharmacist terminal device 40 has a function of receiving prescription data provided from the user terminal device 30 as input, recognizing a medicine prescribed to the user U by the doctor D on the basis of the input prescription data, and providing information on the recognized medicine (hereinafter referred to as “medicine information”) to the information linkage server 50A along with user information on the user U. The user information includes at least identification information of the user U. Further, the pharmacist terminal device 40 has a function of receiving genetic data provided from the genetic information server 10, medical checkup data of the user U provided from the medical checkup information server 20, and the like as inputs and supporting execution of administration instructions performed by the pharmacist M on the user U on the basis of the input genetic data, medical checkup data of the user U, and the like. Further, the pharmacist terminal device 40 has a function of instructing a delivery base L to deliver the medicine to the whereabouts of the user U, or the like.

These functions of the pharmacist terminal device 40 are realized, for example, by the pharmacist terminal device 40 executing an application program (hereinafter referred to as a “pharmacist application”). The pharmacist application may be installed in the pharmacist terminal device 40 as a dedicated application, or may be realized by a general-purpose application such as a browser.

The information linkage server 50A has a function for providing information necessary for online administration instructions for the user U to the pharmacist terminal device 40. Specifically, the information linkage server 50A receives user information and medicine information of the user U as inputs via the pharmacist terminal device 40 as inputs, and manages both the input user information and the medicine information of the user U in association with each other. Further, the information linkage server 50A acquires genetic data regarding a medicine prescribed to the user U from the genetic information server 10 in response to a request of the pharmacist terminal device 40, acquires medical checkup data of the user U from the medical checkup information server 20, and provides the acquired genetic data and medical checkup data of the user U to the pharmacist terminal device 40.

Further, the information linkage server 50A has a function of recording an administration state of the user U with reception of a notification of the administration state of the user U after delivery of the medicine from the user terminal device 30 as a trigger and executing follow-up after delivery of the medicine to the user U on the basis of the notified administration state. For example, the information linkage server 50A checks whether or not the medication of the user U is taken as instructed by the pharmacist M, and if the medication is not taken as instructed by the instruction, performs notification to encourage the user U to take medication or to correct incorrect usage or doses as follow-up. Note that, in a case where the pharmacist M executes follow-up, the information linkage server 50A may provide information necessary for follow-up to the pharmacist terminal device 40.

According to such a configuration, the pharmacist M can provide administration instructions to the user including cautions with respect to side effects of the delivered medicine on the basis of the genetic information and the medical checkup data of the user U in online administration instructions executed for the user U. Further, the pharmacist M can ascertain the administration state (administration state) of the medicine delivered to the user U and provide information for follow-up to the user U as necessary. Since such administration instructions and follow-up can be executed online, the online dispensing system 100A of the embodiment can improve the quality of online administration instructions.

Note that, although one user U is illustrated as a user of the online dispensing system 100A in FIG. 1 for simplicity, this does not mean that a user of the online dispensing system 100A has to be one. There may be a plurality of users of the online dispensing system 100A, and in that case, it is assumed that access to information on each user is appropriately controlled on the basis of identification information of each user, or the like.

The online dispensing system 100A of the embodiment having the above-described effects will be described in more detail below.

### (Configuration of Information Linkage Server)

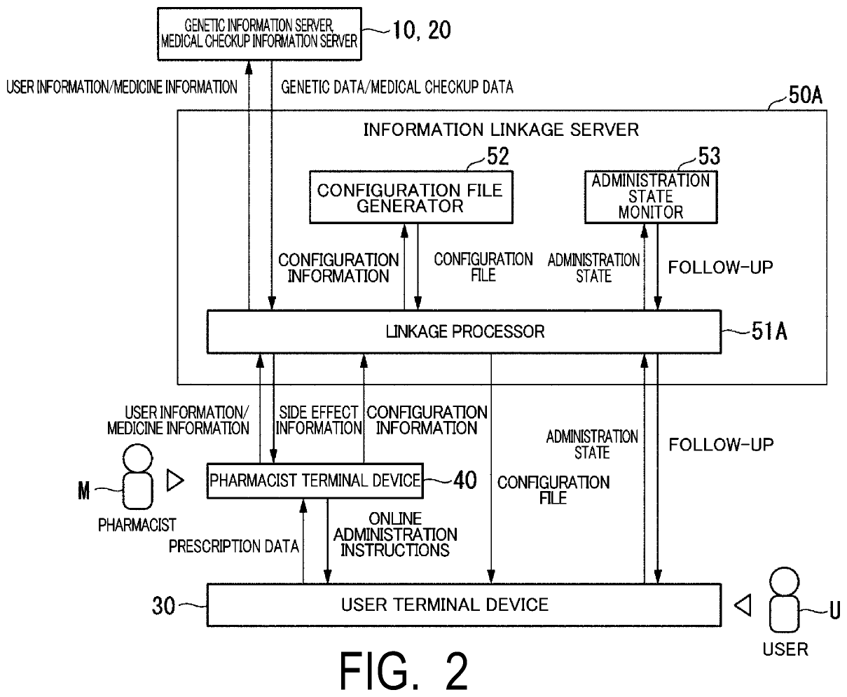

FIG. 2 is a diagram illustrating an example of a functional configuration of the information linkage server 50A. The information linkage server 50A includes, for example, a linkage processor 51A, a configuration file generator 52, and an administration state monitor 53. The linkage processor 51A has a function of linking various types of information among the genetic information server 10, the medical checkup information server 20, the user terminal device 30, and the pharmacist terminal device 40.

More specifically, upon receiving a request for provision of side effect information with respect to the user U from the pharmacist terminal device 40, the linkage processor 51A requests provision of genetic data and medical checkup data from the genetic information server 10 and the medical checkup information server 20 by linking user information and medicine information of the user U to the genetic information server 10 and the medical checkup information server 20 in response to the received request for provision of the side effect information. The linkage processor 51A acquires genetic data and medical checkup data provided from the genetic information server 10 and the medical checkup information server 20 as responses to the request, and provides the acquired genetic data and medical checkup data to the pharmacist terminal device 40 that is a request source as side effect information.

The linkage processor 51A receives configuration information provided from the pharmacist terminal device 40 as input, links the input configuration information to the configuration file generator 52, receives a configuration file provided from the configuration file generator 52 as input, and provides the input configuration file to the user terminal device 30. Here, the configuration file is data for storing information on online administration instructions executed by the pharmacist M in the user terminal device 30. The configuration file is appropriately referred to by the user application. Meanwhile, the configuration information is information necessary to generate the configuration file, and is information indicating the content of administration instructions executed on the user U by the pharmacist M.

For example, the configuration information includes information such as a medicine name, administration timing, a dose of administration, and precautions regarding side effects. Note that the content of the administration instructions executed on the user U are written on the basis of the configuration information and the configuration file and sent to the user along with the medicine. Accordingly, the possibility that the user U recognizes the occurrence of a side effect on the user himself/herself increases, and thus the pharmacist M can find the occurrence of the side effect at an early stage and rapidly report the fact to the doctor D.

The linkage processor 51A acquires information indicating the administration state of the user U from the user terminal device 30 in order to monitor the administration state (administration state) of the medicine of the user U after delivery of the medicine, and records the acquired information indicating the administration state of the user U. For example, when taking the medicine after receiving the delivery of the medicine, the user U inputs information such as the name of the medicine taken, medicine taking time, the dose, and presence or absence of subjective symptoms to the user terminal device 30 as a medication state. The user terminal device 30 notifies the information linkage server 50A of the input medication state. The linkage processor 51A outputs the notified administration state to the administration state monitor 53.

The configuration file generator 52 receives configuration information provided from the linkage processor 51A as input and generates a configuration file on the basis of the input configuration information. The configuration file generator 52 provides the generated configuration file to the user terminal device 30 and manages the configuration file in association with user information of the user U. The configuration file generator 52 is an example of a “setting information generation unit.” The configuration file is an example of “setting information”.

The administration state monitor 53 includes a database (hereinafter referred to as an “administration state database”) that accumulates information indicating medication states of the user U after medicine delivery and performs follow-up after medicine delivery on the basis of the administration states accumulated in the administration state database. For example, the administration state monitor 53 accumulates information such as the name of a medicine taken by the user U, the time when the medicine is taken, the dose of the medicine taken, and presence or absence of subjective symptoms due to medication for each user U as administration states.

Note that, although a case in which provision of the configuration file and notification of the administration state are directly performed between the user terminal device 30 and the information linkage server 50A has been described in the example shown in FIG. 2, provision of the configuration file and notification of the administration state may be performed via the pharmacist terminal device 40.

### (Processing of Genetic Information Server)

Processing executed by the genetic information server 10 is divided into main processing for receiving a request of the information linkage server 50A and providing side effect information in response to the received request and preprocessing executed in advance for the main processing. Specifically, the preprocessing is processing for generating a medicine metabolism list for each user U. The medicine metabolism list is a collection of information on medicine metabolism based on genetic information (hereinafter referred to as “metabolic information”) with respect to various medicines. The genetic information server 10 holds database regarding medicine metabolism of various medicines (hereinafter referred to as a “medicine metabolism database”) in advance, and generates a medicine metabolism list of each user by comparing the medicine metabolism database with the genetic information of the user U. The medicine metabolism list is an example of “list information.”

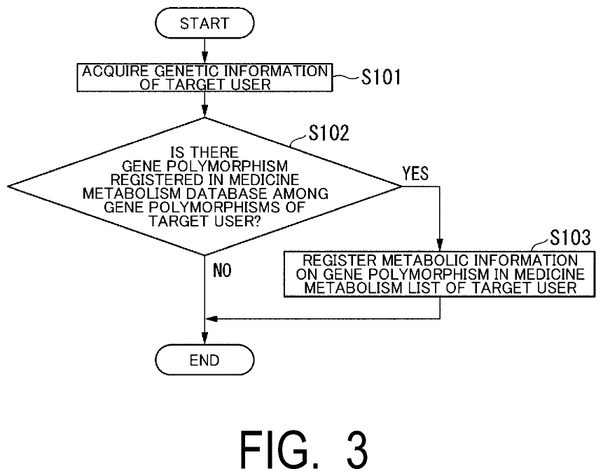

FIG. 3 is a flowchart illustrating an example of a flow of preprocessing performed by the genetic information server 10. The genetic information server 10 executes preprocessing for all users U whose genetic information has been registered therein. For example, the genetic information server 10 may execute the preprocessing at a timing at which genetic information has been registered for a user U whose genetic information has not been registered.

First, the genetic information server 10 acquires genetic information of a user U from genetic information accumulated therein (step S101). The genetic information server 10 determines whether or not there is a gene polymorphism registered in the medicine metabolism database among gene polymorphisms of the user U (step S102). If it is determined that there is a gene polymorphism registered in the medicine metabolism database among the gene polymorphisms of the user U (step S101—YES), the genetic information server 10 registers the registered gene polymorphism, a medicine corresponding to the gene polymorphism, and the content of side effects generated by the medicine in people having the genetic polymorphism in the medicine metabolism list of the user U (step S103), and ends the preprocessing. On the other hand, if it is determined that there is no gene polymorphism registered in the medicine metabolism database among the gene polymorphisms of the user U (step S101-NO), the genetic information server 10 ends the preprocessing without registering metabolic information in the medicine metabolism list of the user U.

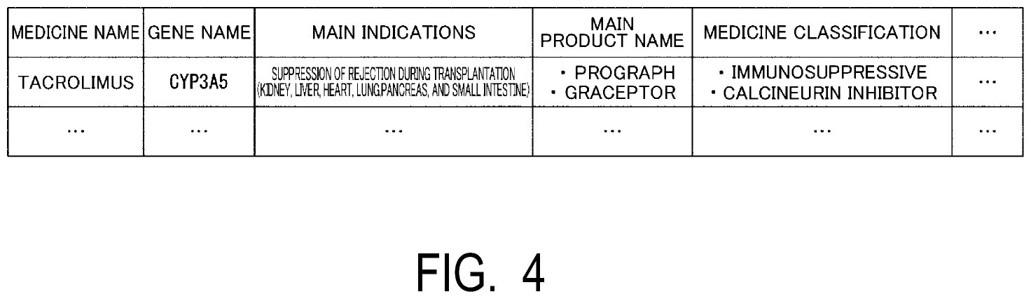

FIG. 4 is a diagram illustrating a specific example of a medicine metabolism list generated through the preprocessing. For example, the medicine metabolism list is generated in the form of a table including a medicine name, an indication, a product name, and items of classification. In this case, classification represents classification of medicines classified according to applications, purposes, and the like. Note that the medicine metabolism list may include other information related to a metabolic rate in addition to the items shown in FIG. 4. The metabolic rate indicates an index of a rate at which a user U related to the medicine metabolism list metabolizes the medicine and is determined on the basis of genetic information of the user U.

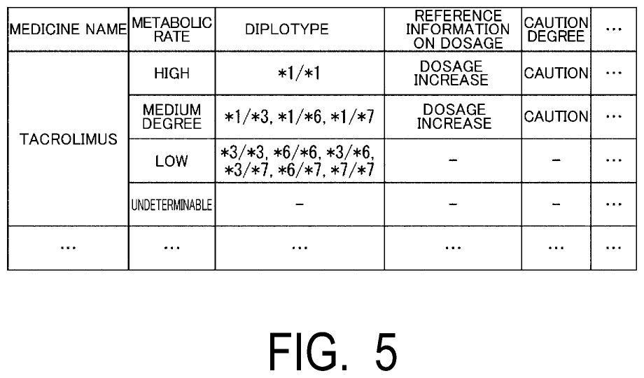

FIG. 5 is a diagram illustrating an example of other information related to a metabolic rate. For example, as related information, information such as a metabolic rate corresponding to a diplotype, reference information related to a dosage, and a caution degree is conceivable. The diplotype is information indicating the type of a genetic polymorphism. The reference information related to a dosage is information mainly for doctors, such as increasing a dosage for a person having a high metabolic rate. The caution degree indicates a degree of caution related to medication or administration of the medicine. In a case where a dosage is increased for a person having a high metabolic rate, for example, the caution degree may be determined from the viewpoint of increasing the possibility of side effects.

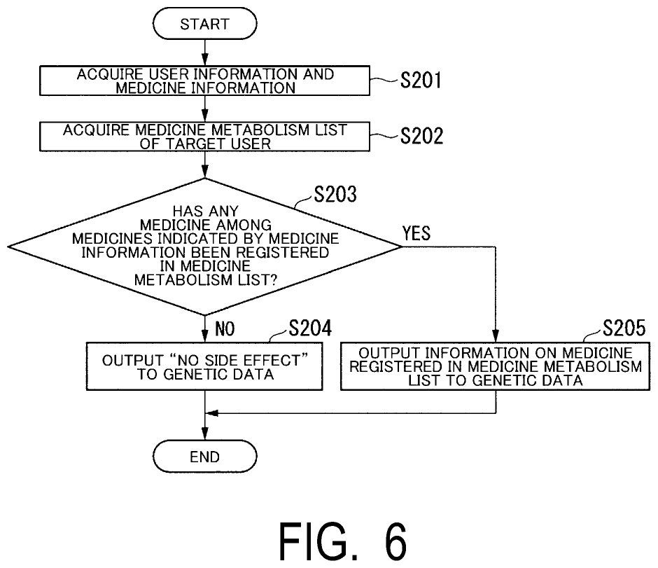

FIG. 6 is a flowchart illustrating an example of a flow of the main processing performed by the genetic information server 10. Specifically, the genetic information server 10 starts the processing flow of FIG. 6 in a case where a request for providing genetic data with respect to a target user U from the information linkage server 50A is received. First, the genetic information server 10 acquires user information and medicine information of the user U from the information linkage server 50A at the time of a request for provision genetic data (step S201). Here, it is assumed that the information linkage server 50A receives a request for provision of side effect information from the pharmacist terminal device 40 and requests provision of genetic data from the genetic information server 10 in response to the received request for provision of the side effect information. It is assumed that the information linkage server 50A acquires user information and medicine information of the user U from the pharmacist terminal device 40 when the request for provision of the side effect information has been received, and provides the user information and the medicine information to the genetic information server 10 at the time of the request for provision of the genetic data.

Subsequently, the genetic information server 10 identifies the user U on the basis of the acquired user information and acquires a medicine metabolism list of the user U from medicine metabolism lists accumulated therein (step S202). The genetic information server 10 determines whether or not any medicine among medicines indicated by the medicine information has been registered in the medicine metabolism list of the user U (step S203). Here, in a case where any one of the medicines indicated by the medicine information is not registered in the medicine metabolism list of the user U (step S203-NO), the genetic information server 10 outputs the fact that there is no side effect (“no side effect”) from the viewpoint of genetic information with respect to the medicine prescribed to the user U to the genetic data (step S204).

On the other hand, in a case where at least one of the medicines indicated by the medicine information has been registered in the medicine metabolism list of the user U (step S203—YES), the genetic information server 10 outputs information on the medicine written in the medicine metabolism list to the genetic data (step S205).

### (Processing of Medical Checkup Information Server)

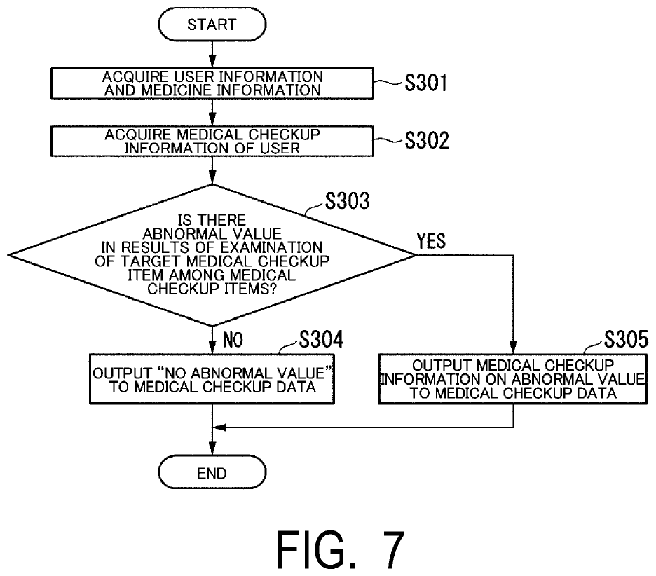

FIG. 7 is a flowchart illustrating an example of a flow of processing for generating medical checkup data by the medical checkup information server 20. Specifically, the medical checkup information server 20 starts the processing flow of FIG. 7 upon receiving a request for providing medical checkup data with respect to a target user U from the information linkage server 50A. First, the medical checkup information server 20 acquires user information and medicine information of the user U from the information linkage server 50A at the time of the request for provision of the medical checkup data (step S301). Here, it is assumed that the information linkage server 50A provides the user information and the medicine information provided from the pharmacist terminal device 40 to the medical checkup information server 20 when the request for provision of the side effect information has been received as in the case of FIG. 6.

Subsequently, the medical checkup information server 20 identifies the user U on the basis of the acquired user information and acquires medical checkup information of the user U from among medical checkup information accumulated therein (step S302). The medical checkup information server 20 determines whether or not an abnormal value is present in examination results of a target medical checkup item among medical checkup items indicated by the medical checkup information of the user U (step S303). For example, the medical checkup information server 20 determines whether or not there is an abnormal value in numerical values with respect to a liver function and a kidney function affecting the ability of medicine metabolism. This is because there is a possibility that the liver function or the kidney function deteriorates due to administration for a long period of time depending on medicines, and by providing such a determination result, the pharmacist M can more safely deliver the corresponding medicine to the user U.

Here, in a case where it is determined that there is no abnormal value in the examination results of the target item (step S303-NO), the medical checkup information server 20 outputs the fact that there is no abnormal value in the medical checkup information of the user U (“no abnormal value”) to the medical checkup data (step S304). On the other hand, in a case where it is determined that there is an abnormal value in the examination results of the target item (step S303—YES), the medical checkup information server 20 outputs medical checkup information with respect to the abnormal value to the medical checkup data (step S305).

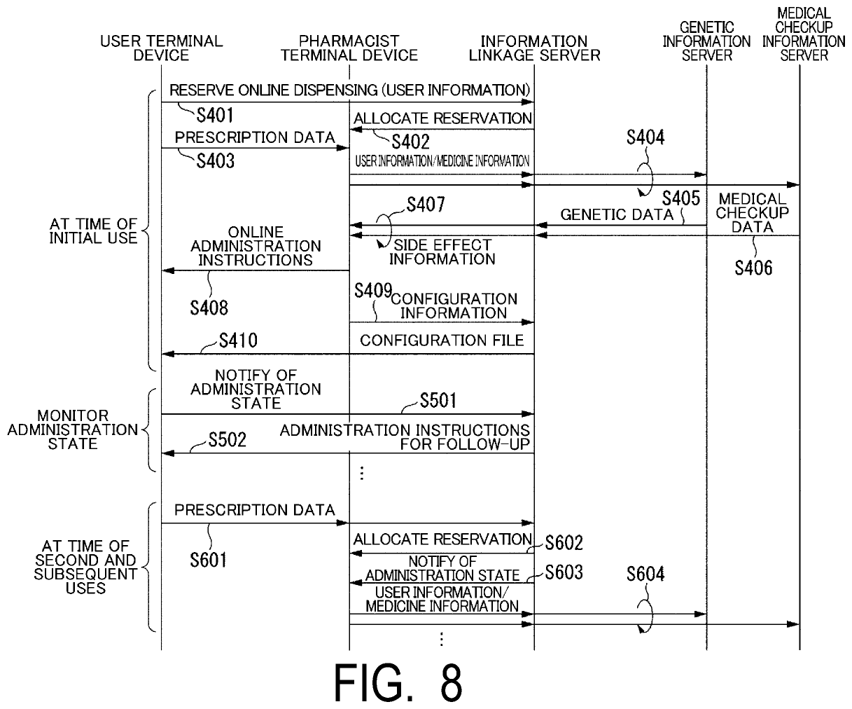

FIG. 8 is a sequence diagram illustrating an example of a flow of processing executed by each server and each terminal device in the online dispensing system 100A with respect to online administration instructions. The series of sequences is executed on the assumption that a user U has received a medical examination of the doctor D and has received an issue of a prescription from the doctor D. Here, it is assumed that the user terminal device 30 has already acquired prescription data from the online medical examination system 60 at the start of the sequence.

First, the user U reserves online dispensing to the information linkage server 50A using the user terminal device 30 (step S401). For example, the user terminal device 30 receives input of various conditions such as a pharmacy where the user U wants to dispense and a timing for receiving online administration instructions at the time of reserving online dispensing, and makes an application for reservation of online dispensing by transmitting the input condition and user information of the user U to an information linkage server 50A.

In response to the reservation application, the information linkage server 50A allocates execution reservation of online dispensing to the user U and transmits reservation information allocated to the user U to the pharmacist terminal device 40 (step S402). For example, the information linkage server 50A previously shares an execution schedule of scheduled online administration instructions with each pharmacy or each pharmacist M, and allocates execution reservation of online administration instructions in consideration of the execution schedule and desired conditions of the user U. The information linkage server 50A may take into account a past online dispensing service use history of the user U and registered position information (for example, an address or the like) at the time of allocating the execution reservation of the online administration instructions.

Upon completion of the execution reservation of online dispensing, subsequently, the pharmacist terminal device 40 acquires prescription data with respect to reserved online dispensing from the user terminal device 30 (step S403). The pharmacist terminal device 40 generates medicine information on the basis of the acquired prescription data and provides the generated medicine information and the user information of the user U to the genetic information server 10 and the medical checkup information server 20 via the information linkage server 50A (step S404). Accordingly, the pharmacist terminal device 40 requests provision of genetic data with respect to the user U from the genetic information server 10 and requests provision of medical checkup data with respect to the user U from the medical checkup information server 20.

Upon receiving the request in step S404, the genetic information server 10 generates genetic data with respect to the user U in response to the received request and provides the generated genetic data to the information linkage server 50A (step S405). Similarly, upon receiving the request in step S404, the medical checkup information server 20 generates medical checkup data with respect to the user U in response to the received request, and provides the generated medical checkup data to the information linkage server 50A (step S406). In the information linkage server 50A, the linkage processor 51A receives the genetic data provided from the genetic information server 10 and the medical checkup data provided from the medical checkup information server 20 as inputs and provides the input genetic data and medical checkup data to the pharmacist terminal device 40 as side effect information (step S407).

Subsequently, the pharmacist terminal device 40 executes online administration instructions for the user U on the basis of the genetic data and the medical checkup data acquired with respect to the user U according to operation of the pharmacist M (step S408). Upon completion of execution of the online administration instructions, the pharmacist terminal device 40 provides configuration information on the online administration instructions to the information linkage server 50A (step S409). In the information linkage server 50A, the configuration file generator 52 receives the configuration information provided from the pharmacist terminal device 40 as input, generates a configuration file on the basis of the input configuration information, and provides the generated configuration file to the user terminal device 30 (step S410).

Note that the configuration file generator 52 may be configured to store the generated configuration file without providing it at that time and to provide the configuration file in a case where a request for provision has been received from the user terminal device 30. Further, the configuration file generator 52 may be configured to perform acquisition of configuration information or provision of a configuration file via the pharmacist terminal device 40.

### (Example of Screen of Pharmacist Application)

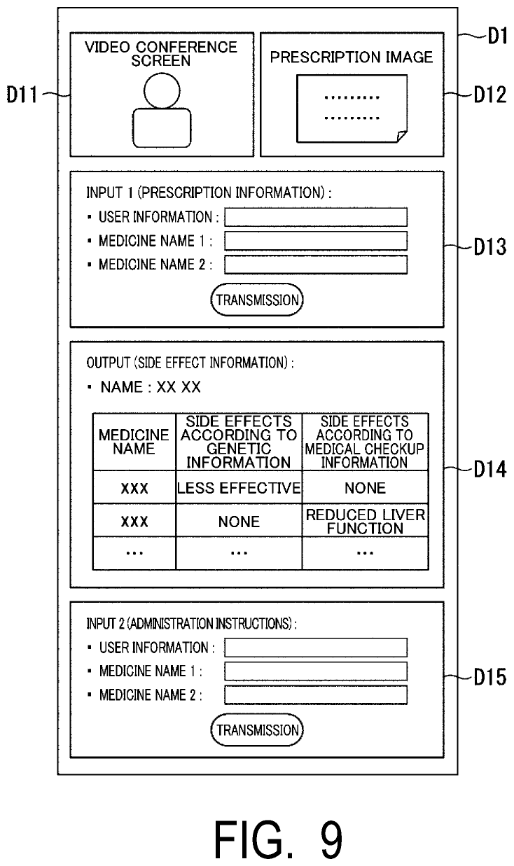

FIG. 9 is a diagram illustrating an example of a screen of a pharmacist application operating in the pharmacist terminal device 40. For example, the pharmacist application causes a display of the pharmacist terminal device 40 to display an administration instruction screen D1 as shown in FIG. 9. The administration instruction screen D1 is a screen used when the pharmacist M executes online administration instructions for the user U. The administration instruction screen D1 includes, for example, a video conference area D11, a prescription image area D12, a prescription information input area D13, a side effect information area D14, and a configuration information area D15. Note that the functions of the respective areas shown in FIG. 9 may be realized by independent applications, or may be configured to be displayed on different screens.

The video conference area D11 is an area in which a screen of a video conference performed with respect to the user terminal device 30 is displayed. For example, the pharmacist M executes online administration instructions for the user U by performing a voice call while displaying a video of the user U in the video conference area D11 using a video conference function of the pharmacist application.

The prescription image area D12 is an area in which an image of a prescription is displayed in a case where image data obtained by imaging the prescription as prescription data is provided from the user terminal device 30. The prescription information input area D13 is an area in which an input interface used by the pharmacist M to input the content (prescription information) of a prescription of the user U is arranged. The prescription information includes medicine information and user information. For example, the pharmacist M inputs the user information and the medicine information to the prescription information input area D13 while viewing a prescription image displayed in the prescription image area D12. Note that manual input of the pharmacist M is not essential in inputting the prescription information. For example, the pharmacist application may read prescription information from a prescription image using a function such as optical character recognition/reader (OCR) and display it in the prescription information input area D13. In this case, the pharmacist application may be configured to receive an input of an operation for correcting read information in the prescription information input area D13. Upon completion of input of the prescription information, the pharmacist M can transmit the input user information and medicine information to the genetic information server 10 and the medical checkup information server 20 via the information linkage server 50A by pressing a transmission button.

The side effect information area D14 is an area in which the content of genetic data provided from the genetic information server 10 and medical checkup data provided from the medical checkup information server 20 with respect to the user U is displayed. The pharmacist M checks the content displayed in the side effect information area D14 at the time of executing online administration instructions for the user U, and instructs a dosage, usage, and the like of medication on the basis of a possibility of side effects of each delivered medicine to the user U.

The configuration information area D15 is an area in which an input interface used to input the content of administration instructions executed by the pharmacist M in online administration instructions for the user U is arranged. For example, the pharmacist M inputs the content of administration instructions to the configuration information area D15 while performing administration instructions for the user U with respect to each medicine during the online administration instructions. Upon completion of input of the content of the instructions, the pharmacist M can transmit the input information to the information linkage server 50A as configuration information by pressing a transmission button.

Return to the description of FIG. 8. In the above steps S401 to S409, the flow of information linkage at the time of initial use of the online dispensing service of the online dispensing system 100A has been described. In the following, information linkage in the case of executing follow-up administration instructions will be described with respect to the user U for which online administration instructions have been executed. Here, it is assumed that the user U appropriately takes the delivered medicine and appropriately inputs administration states to the user terminal device 30 after receiving the online administration instructions. Further, here, it is assumed that the configuration file provided from the information linkage server 50A at the time of initial use has been applied to the user application, and information on the executed administration instructions has been set in the user application in the user terminal device 30.

In this case, the user terminal device 30 notifies the information linkage server 50A of a medication state input by the user U with respect to medication of the user U (step S501). In the information linkage server 50A, the administration state monitor 53 receives the medication state notified from the user terminal device 30 as input, monitors whether the user U correctly takes the medicine on the basis of the input medication state, and executes online administration instructions for follow-up for the user U as necessary (step S502). For example, the administration state monitor 53 may notify the user terminal device 30 of forgetting to take the medicine in a case where an administration state is not notified by a predetermined time with respect to a certain medication timing, or may notify the user terminal device 30 of caution of medication timing in a case where a medication state has been notified at an unexpected timing.

Further, in a case where medication with a wrong dosage or usage has been notified as a medication state, for example, the administration state monitor 53 may point out the error and transmit a notification to encourage the user U to take the medicine at a correct dosage and usage to the user terminal device 30. Further, for example, the administration state monitor 53 may be configured to accumulate medication states notified from the user terminal device 30 and notify the user terminal device 30 of advices, precautions, and the like assumed from a past medication history. For example, the administration state monitor 53 may be configured to notify information such as the total or average of dosages administered for a predetermined period of time with respect to each medicine.

### (Example of Screen of User Application)

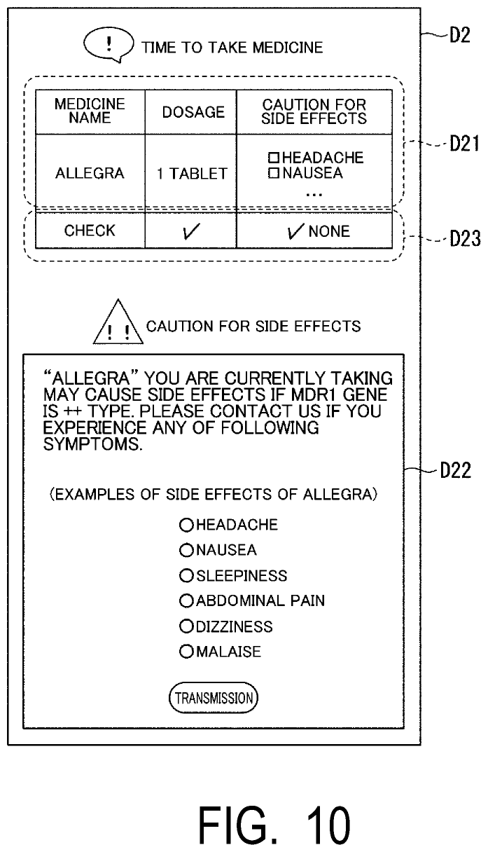

FIG. 10 is a diagram illustrating an example of a screen of the user application operating in the user terminal device 30. For example, the user application causes a display of the user terminal device 30 to display a medication notification screen D2 as shown in FIG. 10. The medication notification screen D2 is a screen for notifying the user U of a timing of taking a medicine. The medication notification screen D2 includes, for example, a medication information display area D21, a side effect information display area D22, and an administration state input area D23. The user application may cause the medication notification screen D2 to be pop-up displayed at a scheduled time of taking a medicine.

The medication information display area D21 is an area in which information regarding a medicine for encouraging the user U to take the medicine (hereinafter referred to as “medication information”) is displayed. The medication information display area D21 shown in FIG. 10 is an example in which medication information for encouraging administration of one-tablet Allegra is displayed. In this case, for example, the user application refers to a configuration file of the user U, identifies a medicine to be taken by the user U at each timing, and displays information such as a dosage and usage with respect to the identified medicine as medication information in the medication information display area D21.

The side effect information display area D22 is an area in which side effect information on a medicine that the user U is encouraged to take is displayed. The side effect information display area D22 shown in FIG. 10 is an example in which, with respect to a user U having MDR1 gene of ++ type, side effect information indicating that side effects such as headache, nausea, drowsiness, abdominal pain, dizziness, and fatigue are likely to occur in a case where a person who has the genotype takes Allegra is displayed. In this case, for example, the user application refers to a configuration file of the user U, checks presence or absence of side effects with respect to a medicine that the user U is encouraged to take, and in a case where there is a side effect, displays the side effect information in the side effect information display area D22.

The administration state input area D23 is an area in which an input interface for allowing the user U to input an administration state of the medicine is arranged. The administration state input area D23 shown in FIG. 10 is an example in which a first check box for checking that an appropriate dosage is taken with respect to a presented medicine, a second check box for checking presence or absence of occurrence of side effects, a transmission button for transmitting check results to the information linkage server 50A, and the like are arranged. In this case, for example, the user application causes the first check box for checking at least a result of medication to be displayed with respect to each medicine that the user U is encouraged to take medication, and in a case there is a side effect, causes the second check box to be displayed in order to check presence or absence of occurrence of the side effect. Further, when the transmission button is pressed, the user application transmits check results to the information linkage server 50A. Note that the information linkage server 50A may be configured to update a configuration file such that it reflects new instruction content and provide the configuration file to the user terminal device 30 in a case where instructions with new content have been performed in follow-up online administration instructions.

Note that, in a case where there are a plurality of medicines to be taken in the medication notification screen D2, the medication information display area D21, the side effect information display area D22, and the administration state input area D23 may be provided for each of the plurality of medicines.

By alternately executing online administration instructions at the time of delivering a medicine and online administration instructions for follow-up as described above, an online dispensing service having a higher added value can be provided to the user U. Note that, although a case in which the administration state monitor 53 compares an administration state notified from the user U with the configuration file of the user U to execute follow-up administration instructions in administration state monitoring is assumed here, the pharmacist M may execute the follow-up administration instructions using the pharmacist terminal device 40 in the same manner as in the first use.

Further, although steps S601 to S604 of the same flow as the first time are shown as a flow of online administration instructions of the second time and thereafter in FIG. 8, online dispensing reservation is omitted in the second online administration instructions. In this case, in a case where the user U uses the same pharmacy as that used first, online dispensing reservation is allocated based on provision of prescription data. As described above, for the second and subsequent use of the online dispensing service, a part of the procedure and processing performed at the time of the first use may be omitted.

Further, in the second online administration instructions, administration state notification (step S603) is added. In this case, the pharmacist terminal device 40 is notified of administration states recorded by the administration state monitor 53 in previous monitoring, and at the time of using the service second time onwards, online administration instructions may be executed in consideration of such information. In this way, the pharmacist M can perform the next and subsequent administration instructions including cautions for side effects likely to change over time, for example, in a case where the user U takes a medicine and produces a side effect even though there is no concern about the side effect in terms of genetic information in past administration instructions, a case where the user concerns about side effects on the basis of updated medical checkup information, and the like.

In the online dispensing system 100A of the first embodiment configured in this manner, the information linkage server 50A has a configuration for providing side effect information based on genetic information of the user U with respect to a medicine prescribed to the user U by performing information linkage with respect to the user U between the genetic information server 10, the medical checkup information server 20, and the pharmacist terminal device 40. According to such a configuration, the information linkage server 50A intervenes in linkage of various types of information, and thus personal information such as genetic data and medical checkup data can be safely distributed. In addition, according to such a configuration, the pharmacist M can provide advice based on genetic information and medical checkup results to the user U with respect to side effects of a medicine to be delivered in online administration instructions, and thus the quality of online administration instructions can be improved.

Specifically, at the time of delivering a medicine prescribed by the doctor D to the user U, the pharmacist M can check a genotype or the like related to medicine metabolism such as CYP with respect to the user U on the basis of side effect information linked through an information linkage server 50A, and perform administration instructions in consideration of a possibility of side effects with respect to the medicine to be delivered. Further, the pharmacist M can check whether or not underlying diseases are overlooked, and especially, with respect to a user U who continues to drink a medicine for a long period of time, whether or not the liver function and the kidney function are affected, and then perform administration instructions by checking medical checkup results of the user U in addition to genetic information. According to such online administration instructions, it is possible to provide merits leading to prevention and early discovery of side effects to the user U, and thus motivation for the user U to receive the online dispensing service can be improved.

### Second Embodiment

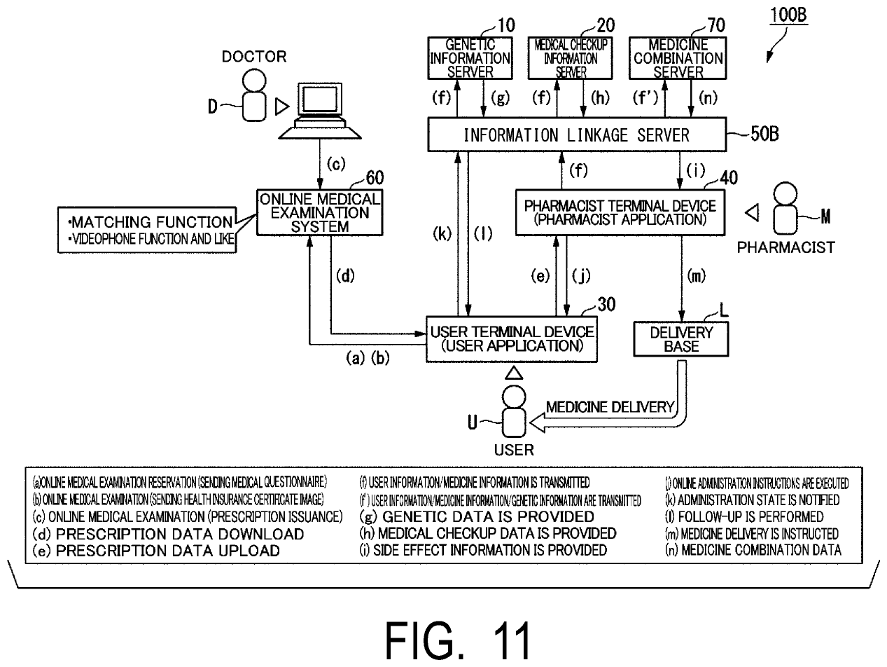

FIG. 11 is a diagram illustrating an example of a configuration of an online dispensing system 100B according to a second embodiment of an administration instruction system of the present invention. The online dispensing system 100B differs from the online dispensing system 100A according to the first embodiment in that the online dispensing system 100B includes an information linkage server 50B in place of the information linkage server 50A and further includes a medicine combination server 70. Note that the configuration of the online dispensing system 100B is the same as that of the online dispensing system 100A except the above. Therefore, the same components are denoted by the same reference signs as in FIG. 1, and description thereof will be omitted here.

The medicine combination server 70 provides information (hereinafter referred to as “medicine combination data”) on possible side effects caused by taking a combination of a plurality of medicines (hereinafter also referred to as “medicine combination”). Here, it is assumed that the medicine combination server 70 holds in advance a database (hereinafter referred to as a “medicine combination database”) with respect to side effects of medicines caused by medicine combination, and information such as side effects due to medicine combination and relevance between side effects and genetic information is accumulated in the medicine combination database. The medicine combination server 70 receives medicine information and genetic information provided via the information linkage server 50B as inputs and searches the medicine combination database on the basis of the input medicine information and genetic information, thereby identifying presence or absence of side effects due to medicine combination with respect to an arbitrary combination of medicines or acquiring the content of side effects. The medicine combination server 70 generates medicine combination data on the basis of such search results and provides the generated medicine combination data to the pharmacist terminal device 40 via the information linkage server 50B. The medicine combination server 70 is an example of a “third information acquisition unit.” Medicine combination data is an example of “third information.”

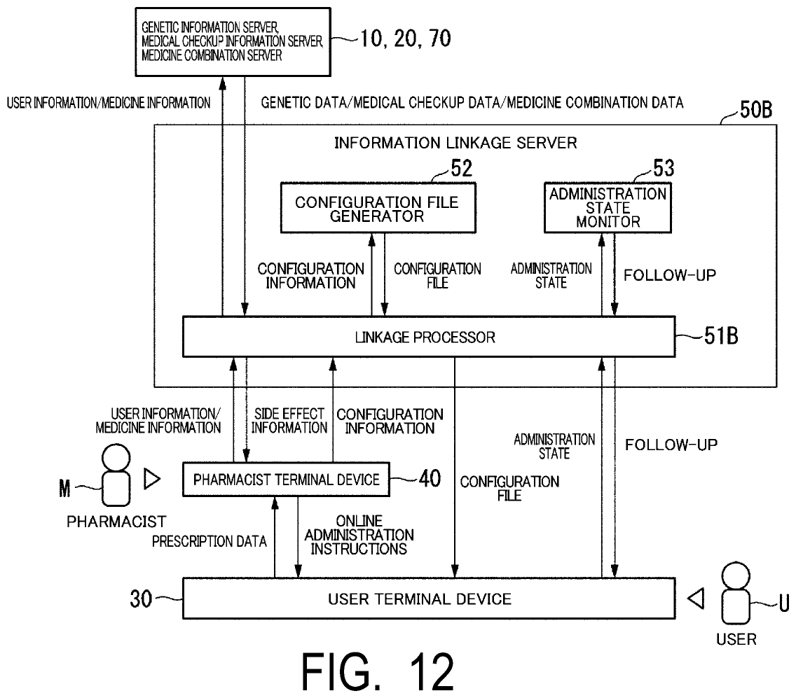

FIG. 12 is a diagram illustrating an example of a functional configuration of the information linkage server 50B. The information linkage server 50B differs from the information linkage server 50A in the first embodiment in that the information linkage server 50B includes a linkage processor 51B in place of the linkage processor 51A. Note that the configuration of the information linkage server 50B is the same as that of the information linkage server 50A except the above. Therefore, the same components are denoted by the same reference signs as in FIG. 2, and description thereof will be omitted here.

The linkage processor 51B has a function of acquiring medicine combination data with respect to medicines prescribed to the user U from the medicine combination server 70 and providing the acquired medicine combination data to the pharmacist terminal device 40 in addition to the function of the linkage processor 51A. More specifically, upon receiving a request for provision of side effect information with respect to the user U from the pharmacist terminal device 40, the linkage processor 51B acquires genetic information of the user U from the genetic information server 10 in response to the received request for provision of the side effect information and provides the acquired genetic information to the medicine combination server 70 along with user information and medicine information. Accordingly, the linkage processor 51B requests provision of medicine combination data from the medicine combination server 70. A request for provision of genetic data and medical checkup data is the same as that in the first embodiment. As a response to such a request, the linkage processor 51B provides genetic data, medical checkup data, and medicine combination data respectively provided from the genetic information server 10, the medical checkup information server 20, and the medicine combination server 70 to the pharmacist terminal device 40 that is the request source as side effect information.

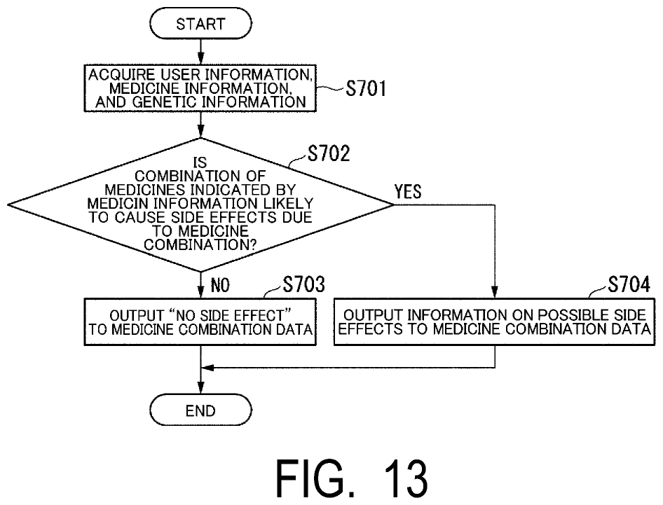

FIG. 13 is a flowchart illustrating an example of a flow of processing executed by the medicine combination server 70. Specifically, the medicine combination server 70 starts the processing flow of FIG. 13 upon receiving a request for provision of medicine combination data with respect to a target user U from the information linkage server 50B. First, the medicine combination server 70 acquires user information, medicine information, and genetic information of the user U from the information linkage server 50B in response to the received request for provision of medicine combination data at the time of receiving the request for provision of the medicine combination data (step S701).

Subsequently, the medicine combination server 70 determines whether or not there is a possibility of causing side effects by medicine combination with respect to a combination of medicines indicated by the medicine information (step S702). In a case where it is determined that there is no possibility of side effects (step S702-NO), the medicine combination server 70 outputs the fact that there are no side effects due to medicine combination (“no side effects”) with respect to medicines prescribed to the user U to the medicine combination data (step S703). On the other hand, in a case where it is determined that there is a possibility of side effects (step S702—YES), the medicine combination server 70 outputs information on possible side effects to the medicine combination data (step S704).

Note that the medicine combination server 70 may be configured to determine whether or not a combination of medicines prescribed to the user U has possible side effects on the basis of past administration states of the user U and people having the same genotype as the user U (hereinafter referred to as “similar users”). For example, the medicine combination server 70 acquires past administration states of the user U or similar users from the administration state monitor 53 of the information linkage server 50B and extracts, from the acquired administration states, combinations of medicines taken in the past by the user U or the similar users and presence or absence of side effects in each combination. The medicine combination server 70 can determine whether or not the combination of the medicines prescribed to the user U is a combination causing side effects for the user U or the similar users in the past by holding in advance a correspondence table in which the extracted combinations of medicines are associated with presence or absence of side effects in each combination. The medicine combination server 70 may include both information obtained on the basis of the medicine combination database and information obtained on the basis of the correspondence table in the medicine combination data.

For example, the medicine combination server 70 may be configured to learn a relationship between combinations of the medicines and presence or absence of side effects by machine learning on the basis of the past administration states of the user U or the similar users and to estimate presence or absence of side effects using learning results. Further, the medicine combination server 70 may be configured to learn a relationship with genetic information of the user U or the similar users in addition to the combinations of the medicines and presence or absence of side effects in the above-described learning of the relationship.

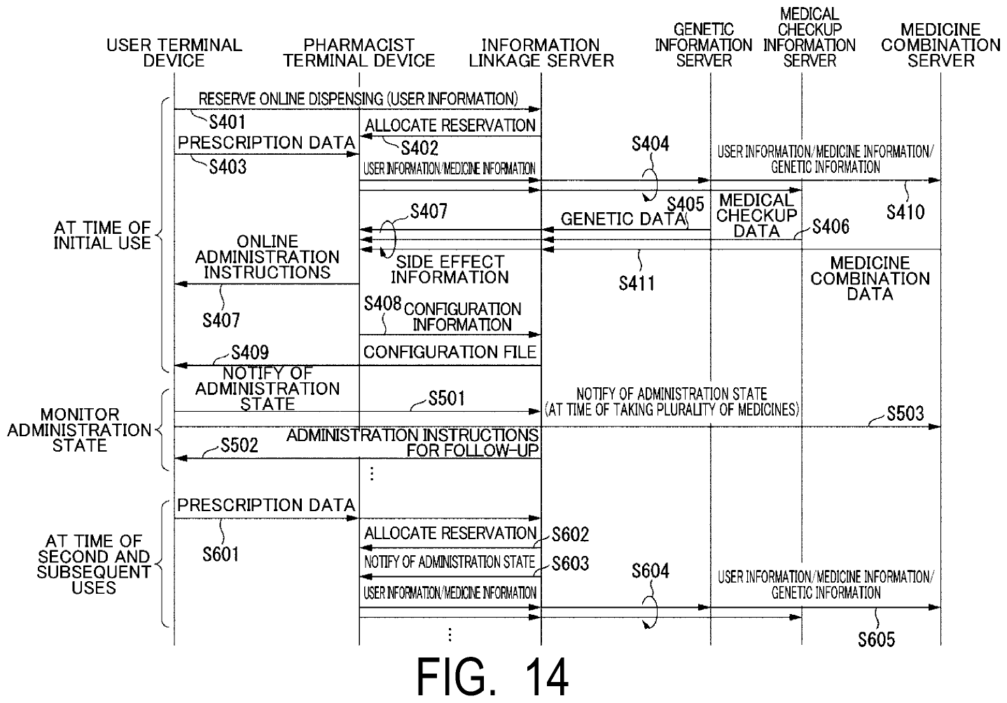

FIG. 14 is a sequence diagram illustrating an example of a flow of processing executed by each server and each terminal device in the online dispensing system 100B with respect to online administration instructions. The flow of processing shown in FIG. 14 differs from the flow of processing in the first embodiment (refer to FIG. 8) in that steps S410 and S411 are added in processing at the time of initial use, step S503 is added in processing at the time of monitoring an administration state, and step S605 is added in processing at the time of second and subsequent use. Here, only differences from FIG. 8 will be mainly described.

Step S410 is processing in which the information linkage server 50B that has received a request for provision of side effect information provides genetic information acquired from the genetic information server 10 along with user information and medicine information to the medicine combination server 70 with the received request for provision of the side effect information as a trigger. Note that step S605 is processing corresponding to step S410 in the second and subsequent use.

Step S411 is processing in which the medicine combination server 70 provides medicine combination data generated on the basis of the genetic information and the medicine information of the user U to the information linkage server 50B. The medicine combination data provided by the information linkage server 50B is provided to the pharmacist terminal device 40 as side effect information along with the genetic data and medical checkup data.

Step S503 is processing of notifying the medicine combination server 70 of an administration state using the user terminal device 30 in a case where the user U takes a plurality of medicines. For example, the user terminal device 30 is configured to notify the information linkage server 50B and the medicine combination server 70 of an input administration state in a case where the user application performs notification of administration timing of a plurality of medicines through the medication notification screen D2 similar to FIG. 10. By notifying of an administration state with respect to medicine combination of the plurality of medicines in this manner, the medicine combination server 70 can accumulate information on medicine combination and presence or absence of side effects with respect to the user U and use the information for learning.

According to the online dispensing system 100B of the second embodiment configured in this manner, in a case where a plurality of medicines to be taken at the same timing are prescribed to the user U, it is possible to further provide side effect information with respect to medicine combination in addition to side effect information based on genetic information and medical checkup information. Therefore, the pharmacist M can execute online administration instructions with higher quality.

### (Modified Examples)

Although a case where the genetic information server 10, the medical checkup information server 20, and the medicine combination server 70 are independent and separate devices has been described in the second embodiment described above, some or all of these servers may be integrated into one server. In this case, a server corresponding to an aggregation destination may be determined according to the security level of each server. For example, the information linkage server 50B acquires genetic information from the genetic information server 10 and provides it to the medicine combination server 70 in the second embodiment, but there is also a way of thinking that it is not preferable to leak the genetic information of the user U to the outside of the genetic information server 10. In such a case, the medicine combination server 70 may be integrated into the genetic information server 10 such that the genetic information is not be provided to the outside.

Although the online dispensing system 100A including both the genetic information server 10 and the medical checkup information server 20 is illustrated in the above first embodiment, the online dispensing system 100A does not necessarily include both the genetic information server 10 and the medical checkup information server 20 and may include at least the genetic information server 10. In this case, the information linkage server 50A may be configured to provide side effect information based on at least the genetic information of the user U to the pharmacist terminal device 40.

Similarly, although the online dispensing system 100B including all of the genetic information server 10, the medical checkup information server 20, and the medicine combination server 70 is illustrated in the second embodiment, the online dispensing system 100B does not necessarily include both the genetic information server 10 and the medical checkup information server 20 and may include at least the genetic information server 10 among them. In this case, the information linkage server 50B may be configured to provide side effect information based on at least genetic data and medicine combination data of the user U to the pharmacist terminal device 40.

The above-described genetic information server 10, medical checkup information server 20, user terminal device 30, pharmacist terminal device 40, information linkage servers 50A and 50B, online medical examination system 60, and medicine combination server 70 may be configured using a processor such as a central processing unit (CPU) and a memory. Each device serves as a device having the above-described function by the processor executing a program. Note that all or part of the configuration of each device may be realized using hardware such as an application specific integrated circuit (ASIC), a programmable logic device (PLD), or a field programmable gate array (FPGA). The aforementioned program may be recorded on a computer-readable recording medium. Computer-readable recording media include, for example, portable media such as flexible disks, magneto-optical discs, ROMs, CD-ROMs, and semiconductor storage devices (for example, a solid state drive (SSD)), and storage devices such as hard disks and semiconductor storage devices built in computer systems. The aforementioned program may be transmitted via telecommunication lines.

Although the embodiment of the present invention has been described in detail with reference to the drawings, a specific configuration is not limited to this embodiment, and design within the scope of the gist of the present invention, and the like are included.

### INDUSTRIAL APPLICABILITY

The present invention is applicable to a system for online delivery of medicines based on a prescription.

### REFERENCE SIGNS LIST

100A, 100B Online dispensing system
10 Genetic information server
20 Medical checkup information server
30 User terminal device
40 Pharmacist terminal device
50A, 50B Information linkage server
51A, 51B Linkage processor
52 Configuration file generator
53 Administration state monitor
60 Online medical examination system
70 Medicine combination server

1-12. (canceled)

13. An online dispensing system comprising:
a first information acquirer configured to acquire first information on side effects of a medicine to be delivered to a user on the basis of medicine information on the medicine written in a prescription of the user and genetic information of the user;
a linkage processor configured to provide the first information as side effect information on the medicine to a presentation information input terminal device used by a presentation information input person who presents information to the user;
a presentation information input terminal device configured to support execution of information presentation through communication with a user terminal device used by the user and to enable reference to the side effect information in the information presentation, with respect to the information presentation of the medicine performed for the user when the presentation information input person delivers the medicine.

14. The online dispensing system according to claim 13, wherein the first information includes metabolic information on medicine metabolism.

15. The online dispensing system according to claim 14, further comprising a medicine metabolism database in which the metabolic information for each medicine is stored,
wherein the first information acquirer acquires the metabolic information on the medicine written in the prescription of the user from the medicine metabolism database, and
the first information includes the acquired metabolic information.

16. The online dispensing method according to claim 14, wherein the metabolic information includes information on at least one of a metabolic rate, a medicine dosage, and necessity of caution.

17. The online dispensing system according to claim 16, wherein the metabolic information includes at least one of information on a medicine metabolism rate, information on a medicine dosage, and information on necessity of caution corresponding to a gene polymorphism of the user.

18. The online dispensing system according to claim 13, wherein the first information acquirer generates in advance, with respect to the user, list information of medicines likely to cause side effects when taken on the basis of genetic information of the user, and generates the first information with respect to medicines included in both the medicine information and the list information.

19. The online dispensing system according to claim 13, further comprising a setting information generator configured to generate setting information for causing the user terminal device to perform an operation according to content of the information presentation performed on the user by the presentation information input person, wherein the linkage processor acquires the content of the information presentation performed by the presentation information input person from the presentation information input terminal device, provides the content to the setting information generator, and provides the setting information generated by the setting information generator to the user terminal device.

20. The online dispensing system according to claim 19, further comprising an administration state monitor configured to monitor an administration state of the medicine from the user in response to a notification from the user terminal device, wherein the setting information causes the presentation information input terminal device to perform an operation of notifying of an administration timing of the medicine, and causes the presentation information input terminal device to perform an operation of notifying the administration state monitor of the administration state of the medicine from the user.

21. The online dispensing system according to claim 20, wherein the administration state monitor provides supplementary information corresponding to the administration state of the user to the user terminal device on the basis of the administration state of the medicine notified from the user terminal device.

22. The online dispensing system according to claim 13, further comprising a second information acquirer configured to acquire second information on side effects of the medicine to be delivered to the user on the basis of the medicine information and medical checkup information of the user,
wherein the linkage processor provides the first information and the second information as the side effect information to the presentation information input terminal device.

23. The online dispensing system according to claim 22, wherein the second information acquirer detects an abnormal value in the medical checkup information of the user and generates the second information on the basis of the detection result.

24. The online dispensing system according to claim 23, wherein the abnormal value is an abnormal value with respect to a kidney function or a liver function.

25. The online dispensing system according to claim 22, further comprising a third information acquirer configured to acquire third information on side effects likely to occur due to medicine combination of a plurality of medicines in a case where the medicine information includes the plurality of medicines prescribed to be taken at the same timing,
wherein the linkage processor provides the first information, the second information, and the third information as the side effect information to the presentation information input terminal device.

26. The online dispensing system according to claim 25, wherein the third information includes information on side effects caused by a combination of past medicines of similar users.

27. The online dispensing system according to claim 26, further comprising a medication state database configured to store information indicating medication states including presence or absence of subjective symptoms due to medication of the user, wherein the third information acquirer acquires, from the medication information database, information on side effects caused by a combination of medicines in past medication of a user having the same genotype as the user with respect to the medicine prescribed to the user and includes the information in the third information.

28. The online dispensing system according to claim 25, wherein the third information includes relevance inference results obtained on the basis of learning results obtained by learning a relevance between combinations of past medicines of similar users and presence or absence of side effects by machine learning.

29. The online dispensing system according to claim 28, wherein the third information acquirer learns a relevance between combinations of medicines and presence or absence of side effects by machine learning on the basis of the genetic information of the user and past administration states of the user and the similar users accumulated in the medication state database, estimates presence or absence of side effects from the learning results, the genetic information and the medicine information of the user, and includes the estimation result in the third information.

30. An information linkage server comprising:
a genetic information server configured to store genetic information of a user;
a presentation information input terminal device used by a presentation information input person who performs information presentation with respect to a medicine to be delivered to the user; and
a linkage processor configured to perform linkage of various types information with a user terminal device used by the user,
wherein the linkage processor acquires medicine information on a medicine written in a prescription of the user from the presentation information input terminal device, provides the medicine information to the genetic information server, and provides first information on side effects of the medicine generated by the genetic information server on the basis of the medicine information to the presentation information input terminal device.

31. The information linkage server according to claim 30, wherein the first information includes information on medicine metabolism.

32. The information linkage server according to claim 31, wherein the linkage processor acquires the metabolic information on the medicine written the prescription of the user from a medicine metabolism database in which the metabolic information for each medicine is stored, and
the first information includes the acquired metabolic information.

33. The information linkage server according to claim 31, wherein the linkage processor further provides third information on side effects caused by a combination of past medicines of similar users to the presentation information input terminal device.

34. The information linkage server according to claim 33, wherein the linkage processor acquires information on side effects caused by a combination of medicines in past medication of a user who has the same genotype as the user with respect to the medicine prescribed to the user from a medication state database that stores information indicating medication states including presence or absence of subjective symptoms due to medication of the user, and includes the information in the third information.

35. An online dispensing method comprising:
acquiring first information on side effects of a medicine to be delivered to a user on the basis of medicine information on a medicine written in a prescription of the user and genetic information of the user; and
providing the first information as side effect information on the medicine to a presentation information input terminal device used by a presentation information input person who presents information to the user,
wherein
the presentation information input terminal device supports execution of the information presentation through communication with a user terminal device used by the user and enables reference to the side effect information in the information presentation with respect to the information presentation of the medicine to be performed on the user when the pharmacist delivers the medicine.

36. A program causing a computer communicating with a genetic information server configured to store genetic information of a user, a presentation information input terminal device used by a presentation information input person who presents information on a medicine to be delivered to the user, and a user terminal device used by the user to:
acquire medicine information on the medicine written in a prescription of the user from the presentation information input terminal device and provide the medicine information to the genetic information server; and
provide information on side effects of the medicine generated by the genetic information server on the basis of the medicine information to the presentation information input terminal device.
